(19) United States
(12) Patent Application Publication
(10) Pub. No.: US 2023/0058010 A1
(43) Pub. Date: Feb. 23, 2023

(54) SYSTEMS AND METHODS FOR DETERMINING THE CONDITION OF MEDICINE FOR USE IN MEDICINE ADMINISTRATION SYSTEMS

(71) Applicant: Medtronic MiniMed, Inc., Northridge, CA (US)

(72) Inventors: Robert A. Vigersky, Washington, DC (US); Yuxiang Zhong, Arcadia, CA (US)

(21) Appl. No.: 17/404,268

(22) Filed: Aug. 17, 2021

(51) Int. Cl. A61M 5/50; A61M 5/24; G06T 7/00; G06K 9/24; G16H 20/13; G16H 20/17

(52) U.S. Cl. CPC A61M 5/5086; A61M 5/2422; G06T 7/0014; G06K 9/24; G16H 20/13; G16H 20/17; A61M 2205/3306; A61M 2205/18; A61M 5/31528

(57) ABSTRACT

A medicine administration system includes a medicine administration device, a medicine cartridge receivable with the medicine administration device, and a contrast background. The medicine cartridge is configured to retain medicine therein. The medicine administration device is configured to dispense at least some of the medicine through a dispensing end of the medicine cartridge. The contrast background is positioned relative to the medicine cartridge to permit visualization of the contrast background through the medicine within the medicine cartridge. The contrast background is configured to facilitate image processing of a captured image of the contrast background through the medicine within the medicine cartridge to determine a condition of the medicine within the medicine cartridge.

### FIELD

The present disclosure relates to medicine administration and, more specifically, to systems and methods for determining the condition of medicine for use in medicine administration systems.

### BACKGROUND

Diabetes mellitus (“diabetes”) is a metabolic disease associated with high blood sugar due to insufficient production or use of insulin by the body. Diabetes affects hundreds of millions of people and is among the leading causes of death globally. Diabetes has been categorized into three types: type 1, type 2, and gestational diabetes. Type 1 diabetes is associated with the body's failure to produce sufficient levels of insulin for cells to uptake glucose. Type 2 diabetes is associated with insulin resistance, in which cells fail to use insulin properly. Gestational diabetes can occur during pregnancy when a pregnant woman develops a high blood glucose level. Gestational diabetes often resolves after pregnancy; however, in some cases, gestational diabetes develops into type 2 diabetes.

Various diseases and medical conditions, such as diabetes, require a user to self-administer doses of medicine. When administering a medicine by injection, for example, the appropriate dose amount is set and then dispensed by the user, e.g., using a syringe, a medicine delivery pen, a pump, etc. In addition to the importance of timely and accurate dosing and dose tracking, particularly for managing lifelong or chronic conditions like diabetes, it is also important to ensure that the condition of the medicine is suitable for use.

### SUMMARY

To the extent consistent, any of the aspects and features detailed herein can be utilized with any of the other aspects and features detailed herein in any suitable combination.

Provided in accordance with aspects of the present disclosure is a medicine administration system including a medicine administration device, a medicine cartridge receivable with the medicine administration device, and a contrast background. The medicine cartridge is configured to retain medicine therein. The medicine administration device is configured to dispense at least some of the medicine through a dispensing end of the medicine cartridge. The contrast background is positioned relative to the medicine cartridge to permit visualization of the contrast background through the medicine within the medicine cartridge. The contrast background is configured to facilitate image processing of a captured image of the contrast background through the medicine within the medicine cartridge to determine a condition of the medicine within the medicine cartridge.

In an aspect of the present disclosure, the contrast background is disposed on or within the medicine cartridge. For example, the contrast background may be disposed on or within an end wall or a side wall of the medicine cartridge.

In another aspect of the present disclosure, the contrast background is disposed on or within a cartridge housing of the medicine administration device. The cartridge housing is configured to receive the medicine cartridge therein. The contrast background, in such aspects, may be disposed on or within an end wall or a side wall of the cartridge housing.

In still another aspect of the present disclosure, the medicine administration device is an injection pen. In such aspects, the injection pen may be configured to electronically track and at least one of store or transmit information relating to an amount of medicine dispensed.

In yet another aspect of the present disclosure, the medicine administration device further includes an image capture device configured to capture an image of the contrast background through the medicine within the medicine cartridge. In such aspects, the medicine administration system may further include a processor configured to process the image to determine a condition of the medicine within the medicine cartridge.

In still yet another aspect of the present disclosure, the image capture device is incorporated into the medicine administration device, is an image capture device of a smart phone (with the processor being a processor of the smart phone or a separate processor), is an image capture device of an accessory configured for use with the medicine administration device (such as, for example, a cap for an injection end of the medicine administration device), or is any other suitable image capture device.

A method of determining the condition of medicine configured for use in a medicine administration system includes capturing an image of a contrast background through medicine within a medicine cartridge, processing the image to determine at least one property of the medicine within the medicine cartridge, and determining a condition of the medicine within the medicine cartridge based upon the determined at least one property.

In an aspect of the present disclosure, the method further includes providing at least one of an alert or a notification indicating the determined condition.

In another aspect of the present disclosure, capturing the image includes capturing the image of the contrast background through a medicine administration device containing the medicine cartridge.

In yet another aspect of the present disclosure, capturing the image is performed by an image capture device separate from the medicine administration device. Alternatively, capturing the image is performed by an image capture device incorporated into the medicine administration device.

In still another aspect of the present disclosure, determining the condition of the medicine within the medicine cartridge includes determining whether the medicine is acceptable for use or not acceptable for use.

### BRIEF DESCRIPTION OF DRAWINGS

FIG. 1A is a schematic illustration of a medicine administration and tracking system configured for use in accordance with the present disclosure including a medicine injection pen, a computing device, and, in aspects, a sensor device and/or a data processing system;

FIG. 1B is a block diagram of the medicine injection pen of the system of FIG. 1A;

FIG. 1C is a block diagram of the computing device of the system of FIG. 1A;

FIGS. 2A and 2B are perspective and longitudinal, cross-sectional views, respectively, of the medicine injection pen of FIG. 1B;

FIGS. 3A and 3B are side, partial longitudinal, cross-sectional views of the medicine injection pen of FIG. 1B with a medicine cartridge thereof in a full condition and the medicine cartridge in a partially emptied condition, respectively;

FIGS. 4A and 4B are side, longitudinal, cross-sectional views of a portion of the medicine injection pen of FIG. 113 with the cartridge housing thereof including a contrast background disposed on or within a side wall thereof and disposed on or within an end wall thereof, respectively;

FIGS. 5A and 5B are side, longitudinal, cross-sectional views of a medicine cartridge configured for use with medicine injection pen of FIG. 1B including a contrast background disposed on or within a side wall thereof and disposed on or within an end wall thereof, respectively;

FIG. 6 is a side, longitudinal, cross-sectional view of an injection end portion of the medicine injection pen of FIG. 4A and an image capture device positioned to capture an image of the cartridge housing against a contrast background;

FIG. 7 is a side, longitudinal, cross-sectional view of the medicine cartridge of FIG. 5A and an image capture device positioned to capture an image of the medicine cartridge against a contrast background;

FIG. 8 is a side, partial longitudinal, cross-sectional view of the medicine injection pen of FIG. 1B including a cap disposed about the medicine cartridge and including image capture components to capture an image of the medicine cartridge against a contrast background;

FIG. 9A is a side, partial longitudinal, cross-sectional view of the medicine injection pen of FIG. 1B including image capture components incorporated therein to capture an image of the medicine cartridge against a contrast background;

FIG. 9B is a bottom end view of the plunger of the medicine injection pen of FIG. 9A illustrating the image capture components thereof;

FIGS. 10A-10C illustrate exemplary contrast backgrounds or background portions configured for use in accordance with the present disclosure; and

FIGS. 11A-11C illustrate images of the contrast backgrounds or background portions of FIGS. 10A-10C, respectively, through medicine to enable detection of various medicine conditions in accordance with the present disclosure.

### DETAILED DESCRIPTION

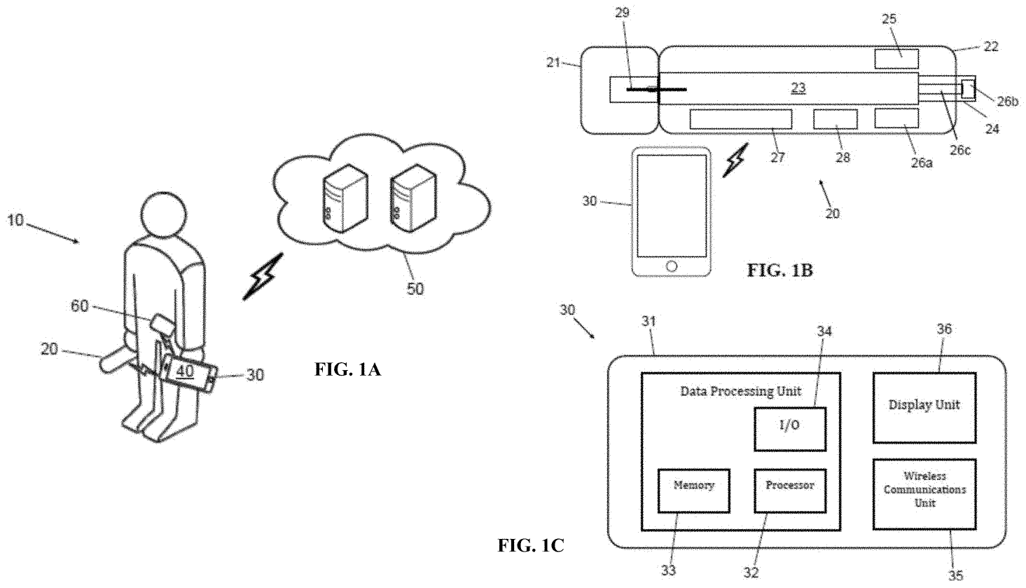

FIG. 1A illustrates a medicine administration and tracking system 10 configured for use in accordance with the present disclosure including a medicine injection pen 20 in wireless communication with a computing device 30 running a health management application 40 associated with pen 20 and/or other devices part of or connected to system 10. System 10, in aspects, further includes a data processing system 50 and/or a sensor device 60. While the present disclosure details a reusable injection pen with replaceable cartridge configured for use as medicine injection pen 20 of system 10 with respect to diabetes management, it is understood that the reusable injection pens of the present disclosure are also applicable to management of other diseases and medical conditions and/or for use with other medicine administration and tracking systems, and/or that disposable injection pens or other medicine administration devices (syringes, pumps, etc.) may also be utilized in accordance with the aspects and features of the present disclosure.

Medicine injection pen 20, described in greater detail below, is a reusable injection pen configured to removably receive a medicine cartridge, e.g., including a vial of insulin (or other suitable liquid medicine), for injecting a selected dose of insulin into a patient and recording information concerning the injected dose of insulin, e.g., a dose amount and/or timestamp data associated with the dose. Medicine injection pen 20 itself or together with computing device 30 and/or other accessory equipment may further be configured to enable determination of the condition of the medicine therein. Alternatively, computing device 30 and/or other accessory equipment may be configured to enable determination of the condition of the medicine within medicine injection pen 20 or may be configured for use in determining the condition of the medicine within a medicine vial configure for use with medicine injection pen 20 (whether installed therein or separate therefrom).

Computing device 30 is detailed and illustrated herein as a smartphone, although any other suitable computing device may be provided such as, for example, a tablet, a wearable computing device (e.g., a smart watch, smart glasses, etc.), a laptop and/or desktop computer, a smart television, a network-based server computer, etc.

Health management application 40 is paired with pen 20, which may be a prescription-only medical device, via smartphone 30, although other suitable configurations are also contemplated. In aspects, the pairing of smartphone 30 with pen 20 at least partially unlocks health management application 40 to enable the user to utilize some or all features of health management application 40, e.g., according to the user's prescription. Thus, the act of pairing can unlock and enable the functionality of health management application 40 and/or system 10 (including pen 20), while health management application 40 (and/or system 10) may provide only limited features in the absence of pairing with pen 20.

Health management application 40 of smartphone 30, in aspects, can monitor and/or control functionalities of pen 20 and provide a dose calculator module and/or decision support module that can calculate and recommend a dose of medicine for the user to administer using pen 20. Health management application 40 provides a user interface, on the user interface of smartphone 30, to allow a user to manage health-related data. For example, health management application 40 can be configured to control some functionalities of pen 20 and/or to provide an interactive user interface to allow a user to manage settings of pen 20 and/or settings for smartphone 30 that can affect the functionality of system 10 (FIG. 1A). Smartphone 30 can additionally or alternatively be used to obtain, process, and/or display contextual data that can be used to relate to the health condition of the user, including the condition for which pen 20 is used to treat. For example, smartphone 30 may be operable to track the location of the user; physical activity of the user including step count, movement distance and/or intensity, estimated calories burned, and/or activity duration; and/or interaction pattern of the user with smartphone 30. In aspects, health management application 40 can aggregate and process the contextual data to generate decision support outputs, e.g., on the user interface, to guide and aid the user in monitoring their condition, using pen 20, and/or managing their behavior to promote treatment and better health outcomes.

In aspects, system 10 further includes a data processing system 50 in communication with pen 20 and/or smartphone 30. Data processing system 50 can include one or more computing devices in a computer system and/or communication network accessible via the internet, e.g., including servers and/or databases in the cloud. System 10 can additionally or alternatively include sensor device 60 to monitor one or more health metrics and/or physiological parameters of the user. Examples of health metric and physiological parameter data monitored by sensor device 60 include analytes (e.g., glucose), heart rate, blood pressure, user movement, temperature, etc. Sensor device 60 may be a wearable sensor device such as a continuous glucose monitor (CGM) to obtain transcutaneous or blood glucose measurements that are processed to produce continuous glucose values. For example, the CGM can include a glucose processing module implemented on a stand-alone display device and/or implemented on smartphone 30, which processes, stores, and displays the continuous glucose values for the user. Such continuous glucose values can be utilized by health management application 40, for example, for displaying health data, in dose calculation and/or decision support, etc.

With reference to FIG. 1B, pen 20 includes a cap 21 configured to protect a medicine dispensing element (e.g., a needle 29 or the dispensing end of medicine cartridge 23) and a body 22 housing the internal operating components of pen 20 and configured to releasable engage the medicine cartridge 23 therein. Pen 20 further includes a dose dispensing mechanism 24 to dispense (e.g., deliver) medicine contained in medicine cartridge 23 out of pen 20 (e.g., through needle 29); a dose setting mechanism 25 to enable the selection and/or setting of a dose of medicine to be dispensed; an operations monitoring mechanism 28 (e.g., including one or more switches, sensors (electrical, optical, acoustic, magnetic, etc.), encoders, etc.) to qualitatively determine that pen 20 is being operated and/or to monitor the operation of pen 20 (e.g., to quantitatively determine an amount of medicine set and/or dosed); and an electronics unit 27 that can include a processor, a memory, a transceiver, and a battery or other suitable power source.

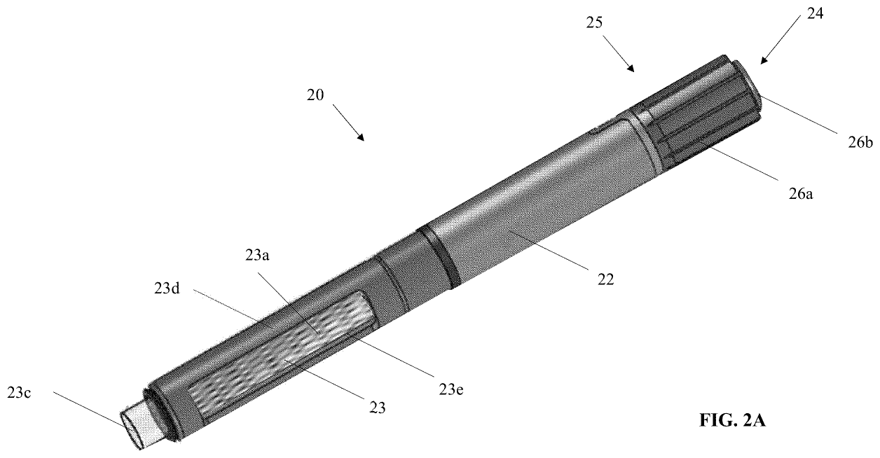

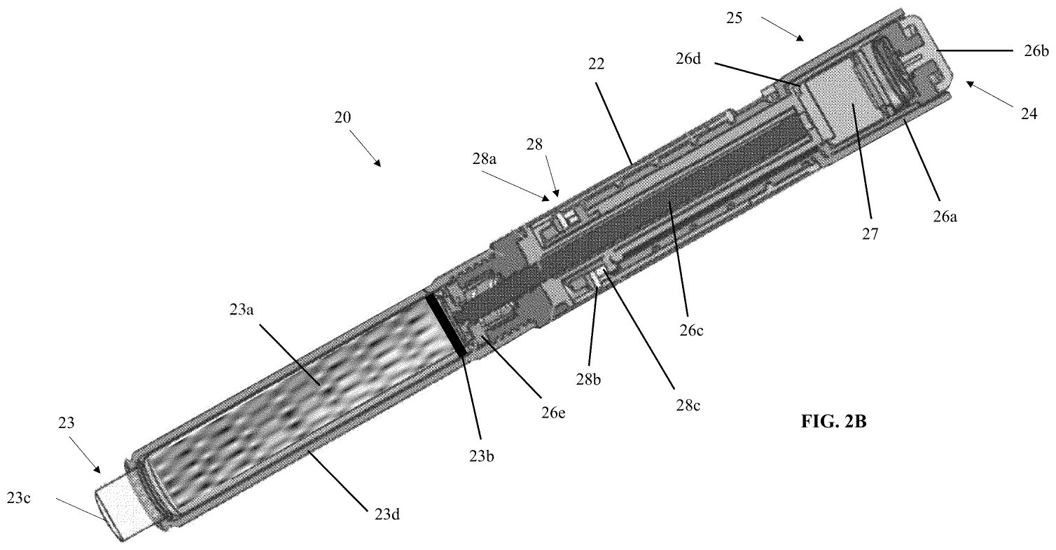

In aspects, in order to operate pen 20, the user first sets e.g., dials, a dose using a dose knob 26a of dose setting mechanism 25. For example, the dose may be adjusted up or down to achieve a desired dose amount prior to administration of the dose by rotating dose knob 26a in an appropriate direction. Once the appropriate dose has been set, the user applies a force against a dose dispensing button 26b of dose dispensing mechanism 24 to begin dispensing. More specifically, to begin dispensing, the user presses against the portion of dose dispensing button 26b that protrudes from body 22 of pen 20 to thereby drive a driving element 26c, e.g., a drive screw 26c, of dose dispensing mechanism 24 against an abutment, e.g., piston 23b (FIG. 2B), of medicine cartridge 23 to dispense an amount of medicine from cartridge 23 through needle 29 into the user in accordance with the dose amount set by dose setting mechanism 25, e.g., dose knob 26a, during setting.

Operations monitoring mechanism 28 of pen 20 senses movement of a rotating and/or translating driving component (e.g., drive screw 26c (see also FIG. 2B)) of dose dispensing mechanism 24. Operations monitoring mechanism 28 may include one or more switches, sensors, and/or encoders for this purpose. More specifically, any suitable switch(es), sensor(s), and/or encoder(s) may be utilized to sense rotary and/or linear movement. Non-limiting examples of such include rotary and linear encoders, Hall effect and other magnetic-based sensors (e.g., magnetic field sensors, magnetic polar sensors, etc.), linearly variable displacement transducers, optical sensors, etc. With respect to an encoder, for example, the encoder can be configured to sense the rotation of drive screw 26c (FIG. 2B) that, in turn, translates to dispense medicine; thus, by sensing rotation of drive screw 26c (FIG. 2B), the translational movement of drive screw 26c can be readily determined. Movement of the encoder may be detected as data processed by the processor of electronics unit 27 of pen 20, from which the amount of medicine dosed can be determined.

In aspects, the processor of electronics unit 27 of pen 20 can store the dose along with a timestamp for that dose and/or any other information associated with the dose. In aspects, the transceiver of electronics unit 27 enables pen 20 to transmit the dose and related information to smartphone 30. In such aspects, once the dose is transmitted, the dose data and any related information associated with that particular transmitted dose is marked in the memory of electronics unit 27 of pen 20 as transmitted. If the dose is not yet transmitted to smartphone 30 such as, for example, because no connection between the pen 20 and smartphone 30 is available, then the dose and associated data can be saved and transmitted the next time a successful communication link between pen 20 and smartphone 30 is established.

The timestamp may be the current time or a time from a count-up timer. When the dose and associated information is communicated to health management application 40 running on smartphone 30, the timestamp and/or “time-since-dose” parameter (as determined by the count-up timer) is transmitted by pen 20 and received by smartphone 30 for storage in memory 33 of data processing unit 31 of the smartphone 30 (see FIG. 1C). Where a count-up timer is utilized, the time of the dose can be determined without pen 20 having to know the current time, which can simplify operation and setup of pen 20. That is, health management application 40 can determined the time of dose based on the current time and the value returned from the count-up timer.

Dose dispensing mechanism 24 of pen 20 can include a manually powered mechanism (user powered and/or mechanically biased), a motorized mechanism, or an assisted mechanism (e.g., a mechanism that operates partly on manual power and partly on motorized power). Regardless of the particular configuration of the dose dispensing mechanism 24, as noted above, when a force (e.g., a manual force, electrically-powered motor force, or combinations thereof) is applied to drive screw 26c of dose dispensing mechanism 24, drive screw 26c turn provides a force to urge medicine from medicine cartridge 23 to deliver the set or dialed dose. In aspects, dose dispensing mechanism 24 can be operated such that rotation and/or translation of the driving element, e.g., drive screw 26c, is facilitated by a variable tension spring or a variable speed motor to inject the dose over a specific time frame (e.g., 1 s, 5 s, etc.) to help reduce the pain of dosing and/or for other purposes.

FIG. 1C illustrates smartphone 30 of system 10 (FIG. 1A) including a data processing unit 31, a wireless communications unit 35, and a display unit 36. Data processing unit 31 includes a processor 32 to process data, a memory 33 in communication with the processor 32 to store data, and an input/output unit (I/O) 34 to interface processor 32 and/or memory 33 to other modules, units, and/or devices of smartphone 30 and/or external devices. Processor 32 can include a central processing unit (CPU) or a microcontroller unit (MCU). Memory 33 can include and store processor-executable code, which when executed by processor 32, configures the data processing unit 31 to perform various operations, e.g., such as receiving information, commands, and/or data, processing information and data, and transmitting or providing information/data to another device. In aspects, data processing unit 31 can transmit raw or processed data to data processing system 50 (FIG. 1A). To support various functions of data processing unit 31, memory 33 can store information and data, such as instructions, software, values, images, and other data processed or referenced by processor 32. For example, various types of Random Access Memory (RAM) devices, Read Only Memory (ROM) devices, Flash Memory devices, and other suitable storage media can be used to implement storage functions of memory 33. I/O 34 of data processing unit 31 can interface data processing unit 31 with wireless communications unit 35 to utilize various types of wired or wireless interfaces compatible with typical data communication standards, for example, which can be used in communications of data processing unit 31 with other devices such as pen 20, via a wireless transmitter/receiver (Tx/Rx), e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, NFC (Near Field Communication), and parallel interfaces. I/O 34 of data processing unit 31 can also interface with other external interfaces, sources of data storage, and/or visual or audio display devices, etc. to retrieve and transfer data and information that can be processed by processor 32, stored in memory 33, and/or exhibited on an output unit of smartphone 30 and/or an external device. For example, display unit 36 of smartphone 30 can be configured to be in data communication with data processing unit 31, e.g., via I/O 34, to provide a visual display, an audio display, and/or other sensory display that produces the user interface of the health management application 40 (FIG. 1A). In some examples, display unit 36 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

Once smartphone 30 receives the dose and related information (e.g., which can include time information, dose setting, and/or dose dispensing information, and other information about pen 20 and/or the environment as it relates to a dosing event), smartphone 30 stores the dose related information in memory 33, e.g., which can be included among a list of doses or dosing events. In aspects, via the user interface associated with health management application 40, smartphone 30 allows the user to browse a list of previous doses, to view an estimate of current medicine active in the patient's body (medicine on board, e.g., insulin on board) based on calculations performed by health management application 40, and/or to utilize a dose calculation module to assist the patient regarding dose setting information on the size of the next dose(s) to be delivered. For example, the patient may enter carbohydrates to be eaten and current blood sugar (which alternatively may be obtained directly from sensor device 60 (FIG. 1A)), and health management application 40 may already know insulin on board. Using these parameters, a suggested medicine dose (e.g., a recommended insulin dose), calculated by the dose determination module, may be determined. In aspects, smartphone 30 can also allow the user to manually enter dose data, e.g., boluses, which may be useful if the battery in pen 20 has been depleted or another medicine delivery device, e.g., a syringe, was utilized to dose.

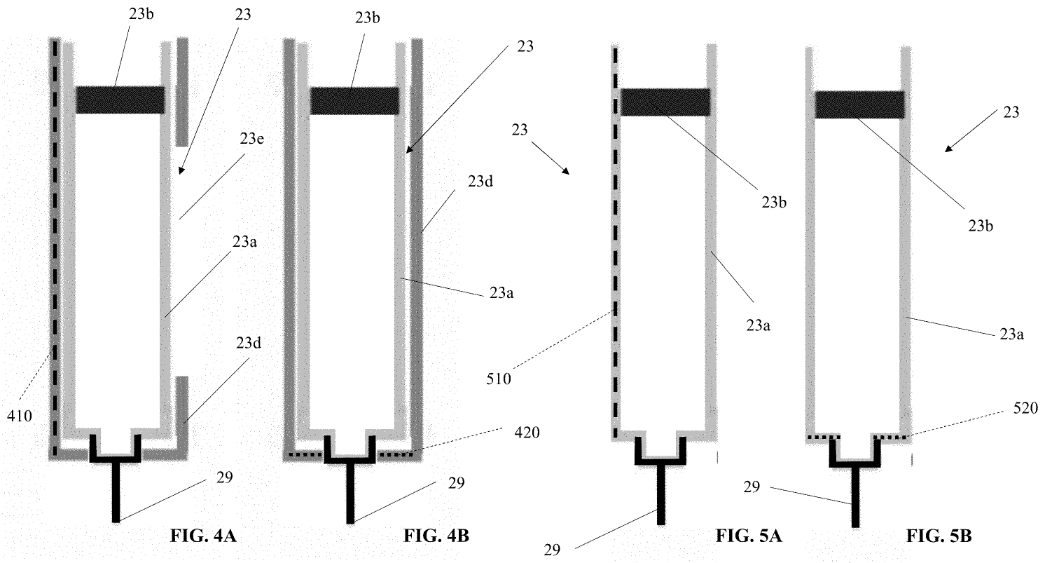

Referring to FIGS. 2A and 2B, pen 20 and, in particular, the mechanical and hardware features thereof, is detailed, although other mechanical and hardware configurations of pen 20 are also contemplated. Pen 20 is shown configured as a reusable device for use with replaceable medicine cartridge 23 which, once emptied (or for other purposes), can be replaced with another medicine cartridge 23 or refilled and reinstalled for subsequent use. Medicine cartridge 23 includes a medicine vial 23a defining an interior volume configured to retain a volume of medicine, e.g., insulin, therein, and a piston 23b sealingly and slidingly disposed within medicine vial 23a such that displacement of piston 23b within medicine vial 23a towards the dispensing end of medicine vial 23a forces medicine from the interior volume through dispensing opening 23c of cartridge 23 and needle 29 (FIGS. 1B, 4A, and 4B) for injection into the user. As can be appreciated, the displacement distance of piston 23b is proportional to the amount of medicine dispensed. Medicine vial 23a is formed at least partially from a transparent material to permit visualization therethrough.

Medicine cartridge 23 is held within a cartridge housing 23d of pen 20 and, in aspects, may be seated within a corresponding cartridge adapter (not shown positionable within cartridge housing 23d to enable use of various different medicine cartridges (e.g., of different size, shape, manufacturer, etc.) with pen 20. Cartridge housing 23d is releasably engageable with body 22 of pen 20, e.g.; via threaded engagement, such that, when cartridge housing 23d is disengaged from body 22 of pen 20, medicine cartridge 23 can be removed and replaced and such that, when cartridge housing 23d is engaged with body 22 of pen 20 with a medicine cartridge 23 therein, medicine cartridge 23 is operably positioned relative to dose dispensing mechanism 24 of pen 20. However, other suitable configurations enabling removal and replacement of a medicine cartridge 23 are also contemplated. In aspects, cartridge housing 23d includes a window 23e defined through a side wall thereof to permit visualization of medicine cartridge 23 when medicine cartridge 23 is disposed within cartridge housing 23d. Window 23e may be a cut-out (without any material therein) to permit visualization therethrough or may be formed from a substantially transparent material to permit visualization therethrough.

Continuing with reference to FIGS. 2A and 2B, dose knob 26a of pen 20 may be coupled to body 22 of pen 20 in threaded engagement via corresponding threads defined on an exterior surface of a portion of dose knob 26a and an interior surface of a portion of body 22. In aspects, electronics unit 27 may reside within an electronics housing disposed or defined within dose knob 26a and be coupled thereto via a locking mechanism 26d (e.g., a catch-protrusion mechanism, a clutch, etc.) such that, when dose knob 26a is rotated into or out of body 22 to select or adjust the dose to be injected, electronics unit 27 remains stationary (e.g., wherein the locking mechanism 26d is in an unlocked state); however, when dispensing button 26b is actuated, locking mechanism 26d is engaged to lock electronics unit 27 and dose knob 26a to one another such that electronics unit 27 and dose knob 26a rotate together as they translate into body 22 upon actuation of dose dispensing mechanism 24 to inject the selected dose.

The rotation of the dose knob 26a (and electronics unit 27) during actuation drives (direct or indirect) rotation of drive screw 26c which rides within a nut 26e which is fixed to body 22 of pen 20. In this manner, rotation of drive screw 26c also results in translation of drive screw 26c (due to the pitched threading of drive screw 26c) towards medicine cartridge 23 to thereby drive piston 23b through medicine vial 23a to expel medicine from medicine cartridge 23 for injection into the user. The extent to which dose knob 26a extends from body 22 of pen 20 prior to actuation (which corresponds to the selected dose to be injected) defines the maximum amount of rotation of dose knob 26a and, thus, drive screw 26c during actuation; as such, the amount of medicine expelled from medicine cartridge 23 during actuation cannot exceed the selected dose amount.

Operations monitoring mechanism 28 of pen 20 may include a rotary encoder 28a having a first part 28b rotationally fixed relative to body 22 of pen 20 and a second part 28c rotationally fixed relative to drive screw 26c such that relative rotation between the first and second parts 28b, 28c (which, in turn, is indicative of rotation of drive screw 26c relative to body 22 during dose dispensing), can be sensed and, thus, from which an amount of medicine dispensed can be determined (due to the proportional relationship between rotation of drive screw 26c and translation of piston 23b). Alternatively or additionally, rotary encoder 28a may be configured to sense the amount of medicine dialed for dosing. In aspects, rotary encoder 28a is an electrical contact encoder including one or more contacts disposed on one of the parts 28b. 28c and a code wheel disposed on the other part 28b, 28c, although other configurations and/or types of encoders are also contemplated. Regardless of the particular type of encoder or other sensory components of operations monitoring mechanism 28, relative motion is measured and transmitted to electronics unit 27 for processing (e.g.; determining an amount of medicine dispensed), storage (e.g., storing in memory the amount of medicine dispensed together with timestamp data) and/or transmission (e.g., transmitting the stored data to smartphone 30).

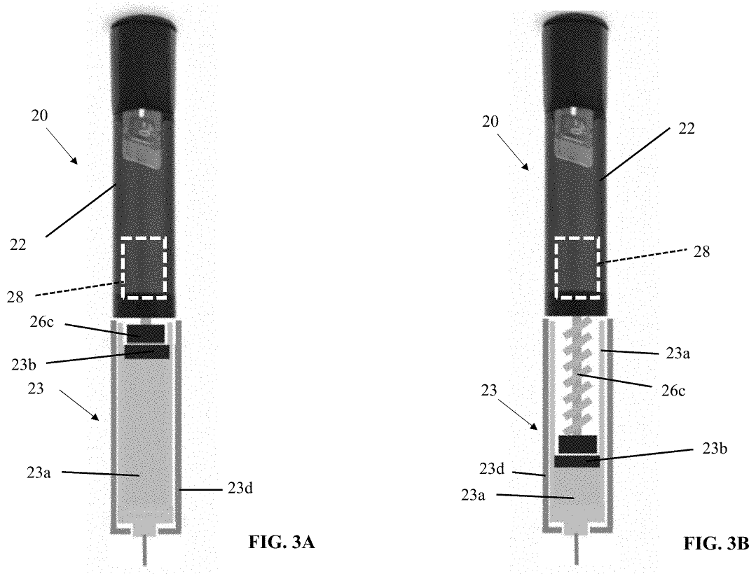

Referring to FIGS. 3A and 3B, as noted above, operations monitoring mechanism 28 detects an amount of rotation of drive screw 26c, which is proportional to the distance drive screw 26c is translated. More specifically, with drive screw 26c in abutment or close proximity to piston 23b prior to activation, the distance drive screw 26c is translated during activation indicates the distance piston 23b is translated which, in turn, is proportional to the amount of medicine dispensed from medicine vial 23a. Thus, the amount of medicine dispensed can be calculated by detecting the amount of rotation of drive screw 26c (without having to know an absolute start or end position of drive screw 26c) and performing the appropriate calculations, e.g., according to the mathematical relationships between drive screw rotation and translation, drive screw translation and piston translation, and piston translation and medicine dispensed. FIG. 3A illustrates pen 20 in an initial condition corresponding to a substantially full condition of cartridge 23, wherein drive screw 26c is disposed in abutment or close proximity with piston 23b, which is disposed at the end of a substantially full medicine vial 23a of cartridge 23. Upon one or more actuations from the initial position shown in FIG. 3A, to a subsequent position shown in FIG. 3B, the detected amount of rotation of drive screw 26c can be used to calculate the amount of medicine dispensed.

Turning to FIG. 4A, in aspects, cartridge housing 23d includes a contrast background 410 disposed thereon or therein. More specifically, contrast background 410 may be disposed on or within an interior surface of a side wall of cartridge housing 23d opposite window 23e. Contrast background 410 extends along at least a portion of the length of medicine vial 23a. Contrast background 410 may include a sheet of material adhered to cartridge housing 23d, may be printed onto the interior surface of cartridge housing 23d, or may be formed on or within cartridge housing 23d in any other suitable manner. Contrast background 410 may include any suitable colors, shades, patterns, images, textures, combinations thereof, etc. In aspects, contrast background 410 includes different portions with each portion including different colors, shades, patterns, images, textures, combinations thereof, etc. such that each portion facilitates detection of one or more properties of medicine condition. Contrast background 410 is visible through window 23e of cartridge housing 23d and medicine vial 23a such that, as detailed below, image-based determination of the condition of the medicine in medicine vial 23a can be performed.

With reference to FIG. 4B, in aspects, as an alternative to cartridge housing 23d including a contrast background 410 disposed on or within a side wall thereof (see FIG. 4A), cartridge housing 23d may include a contrast background 420 disposed on or within an end wall thereof adjacent the injection end thereof. Contrast background 420 may define a generally ring-shaped configuration to surround needle 29. Contrast background 420 may otherwise be configured similar to contrast background 410 (FIG. 4A), as detailed above. In aspects where contrast background 420 is provided, piston 23d may be formed at least partially from a transparent material to permit visualization therethrough and through medicine vial 23a to visualize contrast background 420. Further, in such aspects, window 23e (FIG. 4A) of cartridge housing 23d, may be omitted, although window 23e (FIG. 4A) may still be provided in other aspects. Contrast background 420 enables, as detailed below, image-based determination of the condition of the medicine in medicine vial 23a.

Referring to FIG. 5A, as an alternative or in addition to cartridge housing 23d including a contrast background 410, 420 (FIGS. 4A and 4B), a contrast background 510 may be disposed on or within medicine vial 23a. More specifically, contrast background 510 may be disposed on an interior surface of a side wall of medicine vial 23a, an exterior surface of a side wall of medicine vial 23a, or may be disposed within the side wall of medicine vial 23a. Contrast background 510 may include a sheet of material adhered to medicine vial 23a, may be printed onto medicine vial 23a, or may be formed on or within medicine vial 23a in any other suitable manner. Contrast background 510 may include any suitable colors, shades, patterns, images, textures, combinations thereof, etc. In aspects, contrast background 510 includes different portions with each portion including different colors, shades, patterns, images, textures, combinations thereof, etc. such that each portion facilitates detection of one or more properties of medicine condition. Contrast background 510 is visible through medicine vial 23a and, in aspects, cartridge housing 23d (FIG. 4A) (e.g., through window 23e (FIG. 4A) thereof) such that, as detailed below, image-based determination of the condition of the medicine in medicine vial 23a can be performed.

With reference to FIG. 5B, in aspects, as an alternative to medicine vial 23a including a contrast background 510 disposed on or within a side wall thereof (see FIG. 5A), medicine vial 23a may include a contrast background 520 disposed on or within an end wall thereof opposite piston 23b. Contrast background 520 may define a generally ring-shaped configuration to surround needle 29. Contrast background 520 may otherwise be configured similar to contrast background 510 (FIG. 5A), as detailed above. In aspects where contrast background 520 is provided, piston 23d may be formed at least partially from a transparent material to permit visualization therethrough and through medicine vial 23a to visualize contrast background 520.

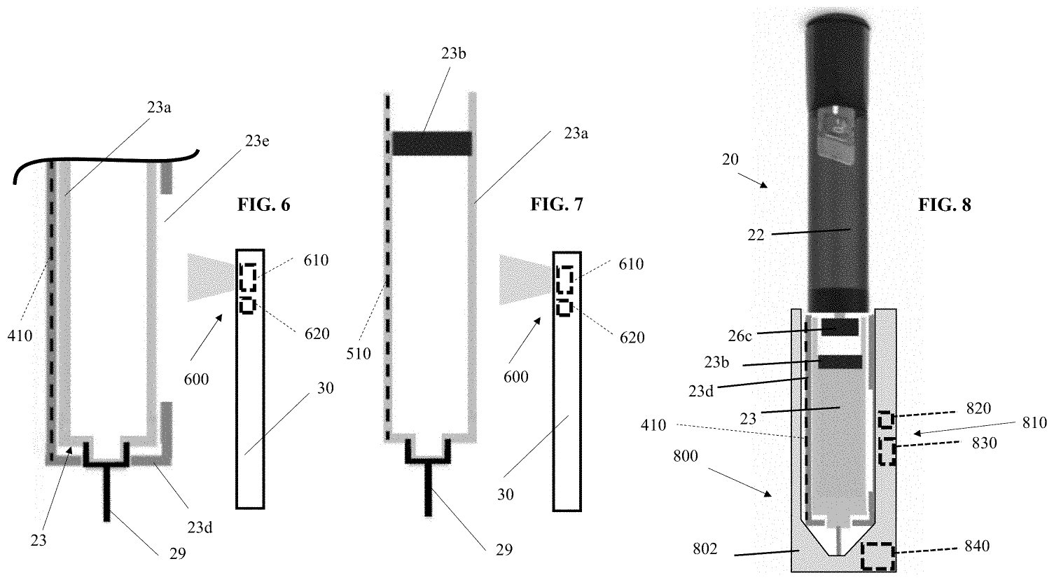

Turning to FIG. 6, the injection end portion of pen 20 is shown together with an image capture device 600, e.g., a camera of smartphone 30 (see also FIG. 1A) for use in illuminating (if necessary) and capturing an image of contrast background 410 through cartridge housing 23d (e.g., window 23e thereof) and medicine vial 23a to detect the condition of the medicine in medicine vial 23a. Medicine condition detection may be performed with cartridge housing 23d (including medicine vial 23a disposed thereon) engaged with housing 22 of pen 20 (see FIGS. 3A and 3B) or disengaged and separate therefrom.

Image capture device 600 may include a camera 610 and a light source 620 to illuminate the field of view of camera 610, if necessary. Although detailed hereinbelow as incorporated into smartphone 30 (FIG. 1A), image capture device 600 may be any suitable general purpose camera (without requiring specialized hardware) such as, for example, the camera of smartphone 30 (FIG. 1A), a camera of a tablet computer, the camera of a laptop computer, a standalone consumer camera, etc. With additional reference to FIG. 1A, the image taken by camera 610 (of contrast background 410 through cartridge housing 23d (e.g., window 23e thereof) and medicine vial 23a) is transmitted to a processor, e.g., processor 32 of the data processing unit of smartphone 30, for image processing. The image processing may be performed by an application running on smartphone 30, e.g., health management application 40 (FIG. 1A) or a separate application. Facilitated by contrast background 410 (and, in aspects, the one or more portions thereof), the image processing performed by processor 32 enables determination of a clarity of the medicine within medicine vial 23a, the presence of particles, fibrils, clumps, other solids, etc. in the medicine within medicine vial 23a, and/or a coloration of the medicine within medicine vial 23a. Additional or alternative properties of the medicine within medicine vial 23a may also detectable. The properties may be assessed on sliding scales (e.g., excellent, good, adequate, poor, very poor), against thresholds (e.g., acceptable versus not acceptable), as measured metrics (e.g., a transparency percentage, a color index, volume of particles, etc.), or in any other suitable manner. As noted above, different portions of the contrast background 410 may be utilized to facilitate determination of one or more different properties. The assessed properties, collectively or individually, are then compared to baseline criteria, e.g., by processor 32 using data stored in memory 33, from which a condition of the medicine within vial 23a is determined by processor 32. The condition may be binary (e.g., acceptable for use versus unacceptable for use), on a sliding scale (e.g., a rating of 1-10), a categorization (e.g., excellent, good, adequate, poor, very poor), a specific determination (e.g., medicine is/is not sufficiently transparent; lack of/presence of fibrils, clumps, etc. in medicine; medicine color appropriate/not appropriate; etc.), or may be provided in any other suitable manner. Further, the condition may be an overall condition of the medicine or a condition of multiple properties of the medicine, e.g., a condition with respect to clarity, a condition with respect to color, a condition with respect to particulate matter, a condition with respect to purity, a condition with respect to the presence of contamination/impurity, a condition with respect to solution suspension, a condition with respect to solution precipitation, a condition with respect to demixing, a condition with respect to discoloration, a condition with respect to distortion due to viscosity changes, a condition with respect to change of refractive index, etc. In aspects, memory 33 stores one or more reference images of the contrast background(s) through medicine of known type and condition(s) to facilitate image processing via comparing the present image to the one or more reference images. The reference images may be pre-stored during manufacturing, uploaded via manufacturer-provided updates, stored from previous condition testing (along with the determined results), or may be provided in any other suitable manner. In other aspects, the type of medicine may be determined by detecting any or all of the above-noted conditions. For example, short-acting insulin, long-acting insulin, and/or intermediate-acting insulin may be distinguished based upon some or all of the above-noted conditions, e.g., intermediate-acting insulins may have buffer substances that render the medicine more cloudy as compared to short-acting insulin and/or long-acting insulin, thus providing at least one ground for distinction.

The result of the condition determination(s) may be communicated to health management application 40 (FIG. 1A) and/or transmitted to pen 20 such that an appropriate alert, lockout, or other suitable action may be taken. For example, where the result of the condition determination(s) indicates that the medicine is acceptable for use, a notification indicating the same may be presented in health management application 40 (FIG. 1A) or provided by pen 20, e.g., via a suitable indicator (visual, audible, etc.). Likewise, where the result of the condition determination(s) indicates that the medicine is not acceptable for use, a notification indicating the same may be presented in health management application 40 (FIG. 1A) or provided by pen 20, e.g., via a suitable indicator (visual, audible, etc.). With respect to condition determination(s) that can be cured, e.g., via shaking the medicine vial 23a, suitable instructions may be provided, e.g., in health management application 40 (FIG. 1A), to enable the user to attempt to remedy the condition. On the other hand, for condition determination(s) that cannot be cured, e.g., where the medicine is no longer safe for use, the user may be prompted by pen 20 or health management application 40 (FIG. 1A) to replace the medicine vial 23a and/or a mechanical lockout of pend 20 may be activated inhibiting use of pen 20 until the medicine vial 23a is replaced.

In aspects, pen 20 and/or health management application 40 (FIG. 1A) may provide alerts for and/or may require (before unlocking pen 20) that the user check the condition of the medicine in medicine vial 23a at periodic intervals; in response to other feedback data (temperature outside upper and/or lower temperature thresholds (as determined by a temperature sensor of pen 20), dropping of pen 20 (as determined by an accelerometer of pen 20), etc.); after prolonged periods without use; after a set number of uses; or in any other suitable manner. In aspects, the alerts/requirements for checking the condition may depend on the type of medicine being used.

FIG. 7 illustrates cartridge 23 removed from pen 20 (FIGS. 2A-2B) together with image capture device 600, e.g., the camera of smartphone 30 (see also FIG. 1A) for use in illuminating (if necessary) and capturing an image of contrast background 420 through medicine vial 23a to detect the condition of the medicine in medicine vial 23a. Medicine condition detection may be performed, and any further action, alerts, etc. provided based thereon similarly as detailed above. With respect to the configuration of FIG. 7, the condition of the medicine can be detected prior to positioning cartridge 23 within pen 20 (see FIGS. 3A and 3B) or after removal of cartridge 23 from pen 20 (see FIGS. 3A and 3B).

FIG. 8 illustrates pen 20 including a cap 800 engaged therewith, e.g., replacing cap 21 (FIG. 1B). Cap 800 includes a body 802 configured to engage cartridge housing 23d, e.g., to protect the injection end of pen 20, and to extend along and enclose at least a portion of a length thereof. Cap 800 includes an image capture device 810 having a camera 820 and a light source 830 to illuminate the field of view of camera 820. Cap 800 further includes an electronics unit 840 that can include a processor, a memory, a transceiver, and a battery or other suitable power source to power and control image capture device 810 and to enable communication of the image captured thereby, e.g., to pen 20 and/or smartphone 30 (FIG. 1A). Image capture device 810 is configured to illuminate (if necessary) and capture an image of contrast background 410 through cartridge housing 23d (e.g., window 23e thereof) and medicine vial 23a to detect the condition of the medicine in medicine vial 23a, where contrast background 410 is provided, or to illuminate (if necessary) and capture an image of contrast background 420 (FIG. 4B) through cartridge housing 23d (e.g., window 23e thereof) and medicine vial 23a, where contrast background 420 (FIG. 4B) is provided. Medicine condition detection may be performed, and any further action, alerts, etc. provided based thereon similarly as detailed above.

With reference to FIGS. 9A and 9B, in aspects, rather than the image capture device being separate from pen 20, an image capture device 900 may be incorporated into pen 20 such as, for example, on a free end portion 902 of drive screw 26c, although other suitable locations are also contemplated. Image capture device 900 includes a camera 910 and a light source 920 to illuminate the field of view of camera 910. Image capture device 900 is configured to illuminate (if necessary) and capture an image of contrast background 420 or 520 (see FIG. 5B) longitudinally through medicine vial 23a to detect the condition of the medicine in medicine vial 23a. Medicine condition detection may be performed, and any further action, alerts, etc., provided based thereon similarly as detailed above.

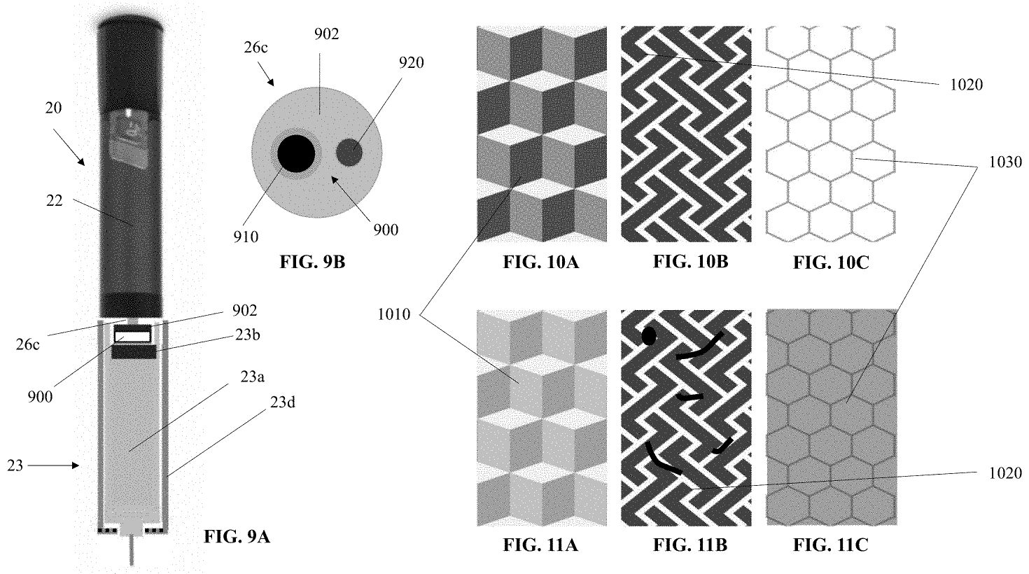

Turning to FIGS. 10A-10C and 11A-11C, as noted above, contrast backgrounds including one or more different contrast background portions are utilized to facilitate detection of one or more properties of medicine to ultimately enable determination of a condition of the medicine. Exemplary contrast backgrounds 1010, 1020, 1030 are illustrated in FIGS. 10A-10C, respectively. Contrast backgrounds 1010, 1020, 1030 may form portions of or the entirety of the contrast backgrounds detailed above. Other suitable contrast backgrounds are also contemplated, as are various different combinations thereof. Indeed, contrast backgrounds 1010, 1020, 1030 are exemplary only and non-limiting.

FIGS. 11A-11C illustrate images, e.g., taken utilizing any of the image capture devices detailed above or any other suitable image capture device, of contrast backgrounds 1010, 1020, 1030 through a medicine vial containing medicine (and, in aspects, through a cartridge housing retaining the medicine vial) to enable detection of various medicine properties as detailed above. As shown via comparison of FIGS. 10A and 11A, the muted brightness and/or sharpness of contrast background 1010 when an image is taken through medicine may facilitate determining a transparency or clarity of the medicine. As shown in FIG. 11B, contrast background 1020 facilitates the identification of particulate (clumps, fibrils, etc.) when an image is taken through medicine. Via comparison of FIGS. 10C and 11C, the shade/color change of contrast background 1030 when an image is taken through medicine may facilitate determining a color or clarity of the medicine.

The various aspects and features disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described functional and/or operational aspects may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing unit” as used herein may refer to any of the foregoing structure or any other physical structure suitable for implementation of the described techniques. Also, the techniques could be fully implemented in one or more circuits or logic elements.

While several aspects of the present disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A medicine administration system, comprising:
a medicine administration device;
a medicine cartridge receivable with the medicine administration device, the medicine cartridge configured to retain medicine therein, wherein the medicine administration device is configured to dispense at least some of the medicine through a dispensing end of the medicine cartridge; and
a contrast background positioned relative to the medicine cartridge to permit visualization of the contrast background through the medicine within the medicine cartridge, the contrast background configured to facilitate image processing of a captured image of the contrast background through the medicine within the medicine cartridge to determine a condition of the medicine within the medicine cartridge.

2. The medicine administration system according to claim 1, wherein the contrast background is disposed on or within the medicine cartridge.

3. The medicine administration system according to claim 2, wherein the contrast background is disposed on or within an end wall or a side wall of the medicine cartridge.

4. The medicine administration system according to claim 1, wherein the contrast background is disposed on or within a cartridge housing of the medicine administration device, the cartridge housing configured to receive the medicine cartridge therein.

5. The medicine administration system according to claim 4, wherein the contrast background is disposed on or within an end wall or a side wall of the cartridge housing.

6. The medicine administration system according to claim 1, wherein the medicine administration device is an injection pen.

7. The medicine administration system according to claim 6, wherein the injection pen is configured to electronically track and at least one of store or transmit information relating to an amount of medicine dispensed.

8. The medicine administration system according to claim 1, further comprising an image capture device configured to capture an image of the contrast background through the medicine within the medicine cartridge.

9. The medicine administration system according to claim 8, further comprising a processor configured to process the image to determine a condition of the medicine within the medicine cartridge.

10. The medicine administration system according to claim 8, wherein the image capture device is incorporated into the medicine administration device.

11. The medicine administration system according to claim 8, wherein the image capture device is an image capture device of a smart phone.

12. The medicine administration system according to claim 11, wherein the processor is a processor of the smart phone.

13. The medicine administration system according to claim 8, wherein the image capture device is incorporated into an accessory configured for use with the medicine administration device.

14. The medicine administration system according to claim 13, wherein the accessory is a cap for an injection end of the medicine administration device.

15. A method of determining the condition of medicine configured for use in a medicine administration system, the method comprising:
capturing an image of a contrast background through medicine within a medicine cartridge;
processing the image to determine at least one property of the medicine within the medicine cartridge; and
determining a condition of the medicine within the medicine cartridge based upon the determined at least one property.

16. The method according to claim 15, further comprising providing at least one of an alert or a notification indicating the determined condition.

17. The method according to claim 15, wherein capturing the image includes capturing the image of the contrast background through a medicine administration device containing the medicine cartridge.

18. The method according to claim 17, wherein capturing the image is performed by an image capture device separate from the medicine administration device.

19. The method according to claim 17, wherein capturing the image is performed by an image capture device incorporated into the medicine administration device.

20. The method according to claim 15, wherein determining the condition of the medicine within the medicine cartridge includes determining whether the medicine is acceptable for use or not acceptable for use.
